(19) United States
(12) Patent Application Publication
(10) Pub. No.: US 2021/0298724 A1
(43) Pub. Date: Sep. 30, 2021

(54) SALIVA COLLECTION DEVICE

(71) Applicant: IUCF-HYU (INDUSTRY-UNIVERSITY COOPERATION FOUNDATION HANYANG UNIVERSITY), Seoul (KR)

(72) Inventors: Kyung-Gyun HWANG, Seoul (KR); Junghwa LEE, Seoul (KR); Youngha SHIN, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 17/265,660

(22) PCT Filed: Aug. 9, 2019
(86) PCT No.: PCT/KR2019/010038

§ 371 Date: Feb. 3, 2021

(30) Foreign Application Priority Data
Aug. 10, 2018 (KR) 10-2018-0093606

(51) Int. Cl. A61B 10/00

(52) U.S. Cl. CPC A61B 10/0051

(57) ABSTRACT

The present invention relates to a saliva collection device. A saliva collection device according to one embodiment of the present invention comprises: a saliva collector part which is provided in a vessel shape open at one side thereof and has an inner collection space open at one side thereof; a saliva movement part having a predetermined length, connected at one end thereof to the saliva collector part, and provided in a pipe structure having a saliva movement passage connected to the collection space; and a saliva collection part connected to the other end of the saliva movement part and having an inner space in which saliva can be stored.

### TECHNICAL FIELD

The present invention relates to a saliva collection device, and more particularly, to a saliva collection device capable of collecting saliva in a pure state while the saliva is not mixed with other substances in an oral cavity.

### BACKGROUND ART

Saliva refers to a secretion fluid secreted into the mouth from salivary glands. More than 99% of the composition of the saliva is water, and the remaining 1% includes electrolytes such as calcium, potassium, and magnesium, proteins, various enzymes, various types of immunoglobulins, antibacterial substances, mucosal glycoproteins, and the like. These substances serve to assist a digestive function, an antibacterial action, and a food softening action, and allow a person to feel a taste. Similar to other body fluids such as blood, urine, and a cerebrospinal fluid, the saliva includes molecules such as proteins and nucleic acids that may reflect pathological and physiological states of the body. In addition, saliva collection is safe, easy, and noninvasive for both inspectors and subjects, so that the saliva collection may be recognized as a very useful disease diagnosis technique.

There is known a scheme in which a person directly spits saliva into a conventional saliva collection device or introduces the saliva into an apparatus. When the saliva is collected by the above scheme, saliva mixed with other substances in an oral cavity, teeth and oral mucosa that are surrounding tissues in the oral cavity, food residues, plaque, normal bacteria, and other bacteria in addition to pure saliva secreted from salivary glands may be collected.

### DISCLOSURE

### Technical Problem

The present invention provides a saliva collection device capable of effectively collecting saliva from an oral cavity of a person.

In addition, the present invention provides a saliva collection device capable of collecting saliva secreted from a specific salivary gland among a plurality of salivary glands located in an oral cavity of a person.

In addition, the present invention provides a saliva collection device capable of preventing saliva from being contaminated during a collection process.

In addition, the present invention provides a saliva collection device capable of preventing tissues from being damaged in a process of collecting saliva.

In addition, the present invention provides a saliva collection device capable of measuring an actual amount of pure saliva secreted from a salivary gland.

### Technical Solution

According to one aspect of the present invention, there is provided a saliva collection device including: a saliva collector part having a container shape having one side that is opened, and formed therein with a collection space having one side that is opened; a saliva movement part having a preset length, having one end connected to the saliva collector part, and having a pipe structure having a saliva movement passage connected to the collection space; and a saliva collection part connected to an opposite end of the saliva movement part, and formed therein with a space in which saliva is stored.

In addition, the saliva collector part may include a connection flow path part formed at a portion of the saliva collector part connected to the saliva movement part, and the connection flow path part may have a front end connected to the collection space and a rear end connected to the saliva movement passage, such that an area of a section perpendicular to a direction from the front end to the rear end gradually decreases from the front end to the rear end.

In addition, an area of a section in the collection space perpendicular to a front-rear direction may be smaller at a rear end of the collection space connected to the saliva movement part than at a front end of the collection space opened toward an outside.

In addition, the saliva collector part may include an attachment part having adhesiveness and provided at a front end of the saliva collector part opened toward an outside.

In addition, the attachment part may be located at the front end of the saliva collector part in a ring shape.

In addition, the saliva collection device may further include an attachment part located on an outer surface of a front end of the saliva collector part that is opened toward an outside, having a ring shape, and having a structure for covering the front end of the saliva collector part.

In addition, the saliva movement part may include a plurality of flow amount measurement markings formed in a length direction thereof.

### Advantageous Effects

According to one embodiment of the present invention, a saliva collection device capable of effectively collecting saliva from an oral cavity of a person in a pure state can be provided.

In addition, according to one embodiment of the present invention, a saliva collection device capable of collecting saliva secreted from a specific salivary gland, such as a parotid gland, among a plurality of salivary glands located in an oral cavity of a person can be provided.

In addition, according to one embodiment of the present invention, a saliva collection device capable of preventing saliva from being mixed with other substances in an oral cavity during a collection process can be provided.

In addition, according to one embodiment of the present invention, a saliva collection device capable of preventing tissues from being damaged in a process of collecting saliva can be provided.

In addition, according to one embodiment of the present invention, a saliva collection device capable of measuring an actual amount of pure saliva secreted from a salivary gland can be provided.

### DESCRIPTION OF DRAWINGS

FIG. 1 is a view showing a saliva collection device according to one embodiment of the present invention.

FIG. 2 is a sectional view showing a front end of the saliva collection device of FIG. 1.

FIG. 3 is an enlarged view showing a portion of a saliva movement part.

FIG. 4 is a sectional view showing a state in which the saliva collection device of FIG. 1 is used.

FIG. 5 is a view showing a front end of a saliva collection device according to another embodiment.

### BEST MODE

According to the present invention, a saliva collection device includes: a saliva collector part having a container shape having one side that is opened, and formed therein with a collection space having one side that is opened; a saliva movement part having a preset length, having one end connected to the saliva collector part, and having a pipe structure having a saliva movement passage connected to the collection space; and a saliva collection part connected to an opposite end of the saliva movement part, and formed therein with a space in which saliva is stored.

### MODE FOR INVENTION

Hereinafter, embodiments of the present invention will be described in more detail with reference to the accompanying drawings. The embodiments of the present invention may be modified into various other forms, and the scope of the present invention should not be construed as being limited to the following embodiments. The present embodiment is provided to more completely describe the present invention to those of ordinary skill in the art. Therefore, in the drawings, a shape of an element has been exaggerated to emphasize a clearer description.

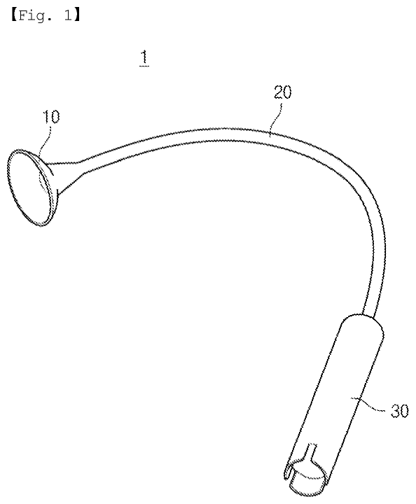

FIG. 1 is a view showing a saliva collection device according to one embodiment of the present invention.

Referring to FIG. 1, a saliva collection device 1 may include a saliva collector part 10, a saliva movement part 20, and a saliva collection part 30.

The saliva collection device 1 may collect saliva from an oral cavity of a person. The saliva is secreted in the oral cavity of the person. The saliva located in the oral cavity is a mixed fluid of saliva secreted from a plurality of salivary glands located in the oral cavity. In the oral cavity, there are large glands called a parotid gland, a sublingual gland, and a submaxillary gland, and other small salivary glands are additionally distributed. The saliva collection device 1 may collect saliva secreted from individual salivary glands located in the oral cavity without being mixed.

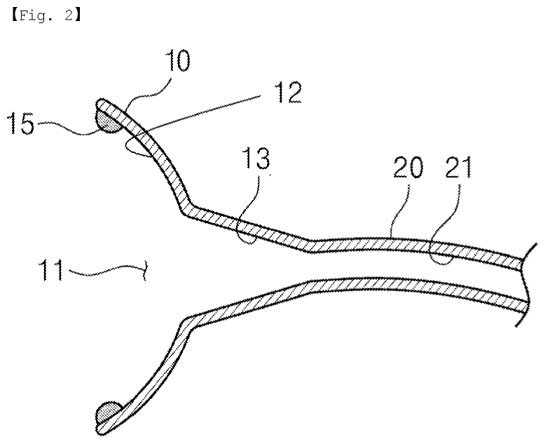

FIG. 2 is a sectional view showing a front end of the saliva collection device of FIG. 1.

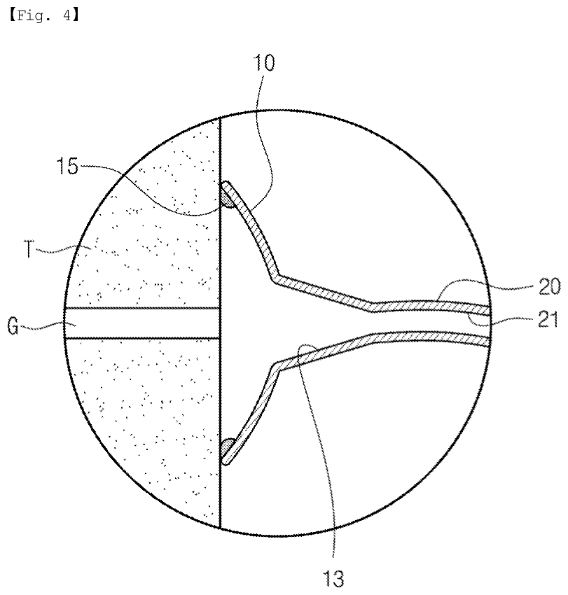

Referring to FIGS. 1 and 2, the saliva collector part 10 may be adjacent to a human biological tissue T (T in FIG. 4) to collect the saliva. The saliva collector part 10 may have a container shape having one side that is opened, and a collection space 11 having one side that is opened may be formed inside the saliva collector part 10. An area of a section in the collection space 11 perpendicular to a front-rear direction may be smaller at a rear end of the collection space 11 connected to the saliva movement part 20 than at a front end of the collection space 11 opened toward an outside. As one example, an inner surface 12 of the saliva collector part 10 may have a shape inclined toward a central region of the collection space 11 from the front end to the rear end.

The saliva collector part 10 may include an attachment part 15. The attachment part 15 may allow the saliva collector part 10 to be fixed to the human body tissue T. The attachment part 15 may be provided at a front end of the saliva collector part 10. The attachment part 15 may have adhesiveness, so that when the front end of the saliva collector part 10 makes contact with the human body tissue T, the attachment part 15 may be attached to the human body tissue T. The attachment part 15 may be located at the front end of the saliva collector part 10 in a ring shape.

One end of the saliva movement part 20 may be connected to a rear end of the saliva collector part 10 to provide a passage through which the saliva collected from the saliva collector part 10 moves. The saliva movement part 20 may have a pipe structure having a preset length, and a saliva movement passage 21 may be formed inside the saliva movement part 20. A section of the saliva movement passage 21 perpendicular to a length direction may have a preset area, so that when the saliva is introduced into the saliva movement passage 21, the saliva may agglomerate with each other due to a surface tension to flow in the form of a droplet. For example, an area of the section of the saliva movement passage 21 may be 0.5 cm² to 2 cm².

A connection flow path part 13 may be formed at the rear end of the saliva collector part 10 connected to the saliva movement part 20. A front end of the connection flow path part 13 may be connected to the collection space 11, and a rear end of the connection flow path part 13 may be connected to the saliva movement passage 21, so that the saliva introduced into the collection space 11 may flow to the saliva movement passage 21. The connection flow path part 13 may be configured such that an area of a section perpendicular to a direction from the front end to the rear end gradually decreases from the front end to the rear end. Accordingly, the formation of air bubbles inside the saliva may be minimized during a process in which the saliva agglomerates in the form of a droplet as the saliva is introduced into the saliva movement passage 21.

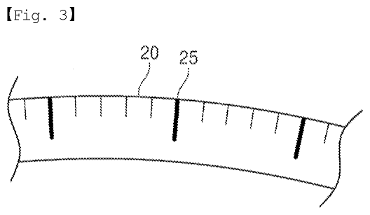

FIG. 3 is an enlarged view showing a portion of a saliva movement part.

Referring to FIG. 3, the saliva movement part 20 may have a flow amount measurement marking 25, and the saliva movement part 20 may be formed of a transparent or translucent material so that the saliva flowing inside the saliva movement part 20 may be visible from the outside. A plurality of flow amount measurement markings 25 may be spaced apart from each other by a preset distance in the length direction of the saliva movement part 20. An amount of the saliva flowing through the saliva movement part 20 may be measured. A distance between adjacent flow amount measurement markings 25 may be set to reflect the amount of the saliva accommodated in the saliva movement passage 21. Accordingly, the amount of the saliva flowing through the saliva movement part 20 may be determined by checking a number of flow amount measurement markings 25 corresponding to a distance between both ends of the saliva accommodated in the saliva movement passage 21. For example, in a case where the distance between the adjacent flow amount measurement markings 25 is set to correspond to 1 ml of saliva located in the saliva movement passage 21, when the distance between the both ends of the saliva accommodated in the saliva movement passage 21 corresponds to two flow amount measurement markings 25, the amount of the saliva may be determined as 2 ml.

The saliva collection part 30 may be connected to an opposite end of the saliva movement part 20 to store the saliva supplied from the saliva movement part 20. The saliva collection part 30 may be formed therein with a space in which the saliva is stored, and may be formed on one side thereof with an opening connected to the saliva movement passage 21. The saliva collection part 30 may be detachably attached to the saliva movement part 20.

FIG. 4 is a sectional view showing a state in which the saliva collection device of FIG. 1 is used.

Referring to FIG. 4, the saliva collection device 1 may be used such that the saliva collector part 10 and a front end of the saliva movement part 20 are inserted into the oral cavity of the person through the mouth, so that the saliva movement part 20 may be adjacent to an oral tissue T of the person. In this case, the saliva collector part 10 may be located to cover a specific salivary gland G from which saliva is collected among a plurality of salivary glands located in the oral cavity. Accordingly, the collection space 11 formed inside the saliva collector part 10 may be shielded from the outside, so that only the saliva secreted from the specific salivary gland G may be collected in the collection space 11, may flow through the saliva movement part 20, and may be collected in the saliva collection part 30.

In addition, through the attachment part 15, the front end of the saliva collector part 10 may be fixed to a circumference of the salivary gland G from which the saliva is collected. Accordingly, while the saliva collector part 10 is stably fixed to the human tissue T in which the salivary gland is located, the collection space 11 may be maintained at a normal pressure. Accordingly, the human tissue T may be prevented from being damaged.

Saliva may be used to determine a physical condition of a person by analyzing the composition thereof. For example, the saliva may be collected, and DNA, RNA, and the like included in the saliva may be studied to identify diseases that are likely to develop in a person who has provided the saliva afterwards. In addition, an amount of secreted saliva may be accurately measured to evaluate a function of a salivary gland from which the saliva is secreted. However, food residues, bacteria, and the like exist in an oral cavity of a person. Such an oral environment may act as a contamination source for the saliva, so that a probability of causing errors in analysis using the saliva collected from the oral cavity of the person may be increased. In addition, when the person has an oral disease, it is difficult to specify a position of the oral disease or to determine a progression state of the disease by using a result obtained by collecting and analyzing saliva secreted from a plurality of salivary glands and mixed with other substances in the oral cavity.

Meanwhile, according to one embodiment of the present invention, the saliva collection device 1 may directly collect the saliva from a periphery of the tissue T in which the specific salivary gland G is located. Accordingly, the salivary gland from which the saliva is collected may be specified, and the saliva may be prevented from being contaminated during the collection process.

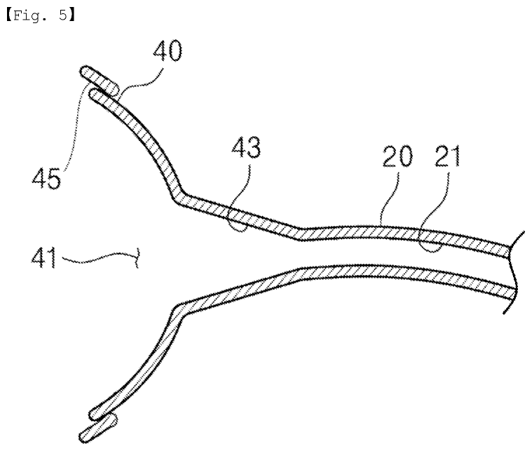

FIG. 5 is a view showing a front end of a saliva collection device according to another embodiment.

Referring to FIG. 5, an attachment part 45 may be provided at a front end of a saliva collector part 40. The attachment part 45 may be located on an outer surface of the front end of the saliva collector part 40. The attachment part 45 may have a ring shape, and may have a structure for covering the front end of the saliva collector part 40. The attachment part 45 may be configured in the form of a soft patch having adhesiveness so as to be attached to a human tissue.

In addition to the structure of the attachment part 45, a configuration and a function of the saliva collector part 40 having a collection space 41 and connected to the saliva movement part 20 through a connection flow path part 43 are the same as in the saliva collection device 1 of FIG. 1, so that redundant descriptions thereof will be omitted.

The above detailed description of the present invention has been provided for illustrative purposes. In addition, preferred embodiments of the present invention are shown and described in the above description, and the present invention may be used in various other combinations, modifications, and environments. In other words, changes or modifications can be made within the scope of the concept of the invention disclosed in the present disclosure, the scope equivalent to the above-disclosed contents, and/or the scope of the skill or knowledge in the art. The above embodiments describe the best mode for implementing the technical idea of the present invention, and various changes required in the specific application fields and uses of the present invention can be made to the embodiments. Therefore, the detailed description of the invention is not intended to restrict the present invention to the disclosed embodiments. In addition, the appended claims should be construed as encompassing other embodiments.

### INDUSTRIAL APPLICABILITY

The saliva collection device according to the present invention may be used to directly collect saliva from a periphery of a tissue in which a salivary gland is located.

1. A saliva collection device comprising:
a saliva collector part having a container shape having one side that is opened, and formed therein with a collection space having one side that is opened;
a saliva movement part having a preset length, having one end connected to the saliva collector part, and having a pipe structure having a saliva movement passage connected to the collection space; and
a saliva collection part connected to an opposite end of the saliva movement part, and foamed therein with a space in which saliva is stored.

2. The saliva collection device of claim 1, wherein the saliva collector part includes a connection flow path part formed at a portion of the saliva collector part connected to the saliva movement part, and
the connection flow path part has a front end connected to the collection space and a rear end connected to the saliva movement passage, such that an area of a section perpendicular to a direction from the front end to the rear end gradually decreases from the front end to the rear end.

3. The saliva collection device of claim 1, wherein an area of a section in the collection space perpendicular to a front-rear direction is smaller at a rear end of the collection space connected to the saliva movement part than at a front end of the collection space opened toward an outside.

4. The saliva collection device of claim 1, wherein the saliva collector part includes an attachment part having adhesiveness and provided at a front end of the saliva collector part opened toward an outside.

5. The saliva collection device of claim 4, wherein the attachment part is located at the front end of the saliva collector part in a ring shape.

6. The saliva collection device of claim 1, further comprising an attachment part located on an outer surface of a front end of the saliva collector part that is opened toward an outside, having a ring shape, and having a structure for covering the front end of the saliva collector part.

7. The saliva collection device of claim 1, wherein the saliva movement part includes a plurality of flow amount measurement markings formed in a length direction thereof.
